(19) United States
(12) Patent Application Publication
(10) Pub. No.: US 2018/0137771 A1
(43) Pub. Date: May 17, 2018

(54) SIMULATED REALITY REHABILITATION SYSTEM

(71) Applicant: Virtual Rehab, Inc., Fresh Meadows, NY (US)

(72) Inventors: Raji Wahidy, Fresh Meadows, NY (US); Mark T. Fulks, Danville, CA (US); Pankaj K. Jain, San Jose, CA (US)

(21) Appl. No.: 15/726,299

(22) Filed: Oct. 5, 2017

Related U.S. Application Data
(60) Provisional application No. 62/422,192, filed on Nov. 15, 2016.

(51) Int. Cl. G09B 19/00; G06N 99/00; G06N 5/02; G09B 9/00; A61B 3/113; A61B 5/021; A61B 5/0402; A61B 5/0476; G06F 3/01

(52) U.S. Cl. CPC G09B 19/00; G06N 99/005; G06N 5/022; G09B 9/00; A61B 3/113; G06T 11/60; A61B 5/0402; A61B 5/0476; G06F 3/011; A61B 2505/09; A61B 5/021

(57) ABSTRACT

The disclosed embodiments include a method performed by a server computer system of a simulated reality platform for rehabilitating users of the platform by promoting real-world behaviors of users engaged with simulations. The method can include initiating a session for a simulated real-world experience that can promote a real-world behavior of a user. The server computer system can receive inputs obtained during the simulated real-world experience, causing a next real-world scene to render in the simulated real-world experience, evaluate the user based on the received inputs processed by expert system to predict the user's real-world behavior and identify a corresponding treatment. Responsive to determining that the user demonstrated corrected or improved behavior based on the evaluation of the user, the server computer system can award the user an opportunity to advance to another scene or level of the session and/or award redeemable points to the user.

### CROSS REFERENCE TO RELATED APPLICATIONS

This application claims the benefit of U.S. Provisional Patent Application No. 62/422,192, filed Nov. 15, 2016, which is incorporated herein in its entirety.

### TECHNICAL FIELD

The disclosed teachings generally relate to a simulated reality system. The disclosed teachings more particularly relate to a virtual or augmented reality system that can rehabilitate users with simulations including real-world scenes that can promote real-world behaviors of users engaged in simulated real-world experiences.

### BACKGROUND

Every person in life typically seeks a second chance. Inmates and substance addicts are no exception. In fact, they are the ones that are in most dire need for help, support, and development to become improved citizens upon their release from prisons or rehabilitation (“rehab”) centers. This is realized through correctional and rehabilitation programs that will prepare them to lead their future lives in a positive manner in order to avoid the possibility of repeated offenses and substance addictions.

According to the International Centre for Prison Studies, the global prison population total is currently set at 10.5 million. Prison budgets are also currently set at roughly $35.2 billion worldwide. From a rehabilitation perspective, there are approximately 255 million suffering from substance abuse and roughly $100 billion are being spent on addiction treatment worldwide. These numbers are enormous and costly to governments, tax payers, and society as a whole. Thus, there is a need for an effective rehabilitation technique that could increase persons treated and reduce costs.

Current techniques for rehabilitating persons are burdensome and typically ineffective. For example, rehab programs require counseling, medication, and/or constant monitoring that is cost-prohibitive. As such, many persons who could be successfully rehabilitated are never treated because of a lack of resources. Moreover, existing techniques for rehabilitating a person fail to provide any insights into whether those techniques are effective in real-time while the persons are being rehabilitated. Instead, a rehabilitation treatment is only deemed successful if a patient can stop being treated without relapsing. Accordingly, a need exists for a personal and a cost-effective rehabilitation system that can also be used to assess and treat persons in real-time.

### SUMMARY

The disclosed embodiments include at least one method performed by a server computer system of a simulated reality platform for rehabilitating users of the platform by promoting real-world behaviors of users engaged with simulations. The method can include initiating a session for a simulated real-world experience including a real-world scene selected from a plurality of real-world scenes. The simulated real-world experience can promote a real-world behavior of a user engaged with the simulated real-world experience. The method can include receiving inputs obtained during the simulated real-world experience, where the inputs can include user responses to prompts, real-world positional data of the user, and/or real-world physiological data of the user. The method can include causing a next real-world scene to render in the simulated real-world experience, where the next real-world scene is selected based on at least some of the received inputs. The method can further include evaluating the user based on the received inputs processed with an expert system to predict the user's real-world behavior and identify a treatment. Responsive to determining that the user demonstrated corrected or improved behavior based on the evaluation of the user, the server computer system can award the user an opportunity to advance to another scene or level of the session and/or award redeemable points to the user. In some embodiments, the server computer system can output data indicative of the evaluation, the predicted user's real-world behavior, and/or a recommendation based on the treatment.

Embodiments include a server computer system of a simulated reality platform for rehabilitating users of the platform by promoting real-world behaviors of users engaged with simulations. The server computer system includes processor(s) and memor(ies) including instructions executable by the processors causing the server computer system to perform certain actions. Those actions can include initiating an augmented reality or virtual reality simulation of a real-world scene selected from a number of scenes, where the selected scene can promote a real-world behavior of a user engaged with the simulation. The server computer system can be further caused to receive inputs obtained during the simulation, where the received inputs include any combination of user responses to prompts, real-world positional data of the user, and/or real-world physiological data. The server computer system can cause a next scene to render in the selected scene, where the next scene is selected based on at least some of the plurality of inputs. The server computer system can also evaluate the user based on the received inputs processed with an expert system to predict the user's real-world behavior and identify a corresponding treatment, output data indicative of the evaluation, the predicted user's real-world behavior, and/or the treatment, and execute machine learning based on the received inputs to improve the expert system for simulations that promote real-world behaviors and identifying treatments or predicting real-world behaviors.

Embodiments include a computer system including processor(s) and memor(ies) including instructions executable by the processors causing the computer system to perform certain actions. These actions can include loading an augmented reality or virtual reality simulation of a real-world scene selected from many real-world scenes, where the selected scene can promote a real-world behavior of a user engaged with the simulation. The computer system can be caused to receive inputs obtained during the simulation, where the received inputs include user responses to prompts, real-world positional data of the user, and/or real-world physiological data.

The computer system can then send the received inputs over a computer network to a server computer system that can enable selection of a next scene by a third-party provider of the simulation to promote the user's real-world behavior, evaluate the user with an expert system to predict the user's real-world behavior and identify a corresponding treatment, output data indicative of the evaluation, the predicted user's real-world behavior, or the treatment, execute machine learning to improve the expert system to promote real-world behaviors, predict real-world behaviors of any users or identify treatments, and load the next scene in the selected scene of the simulation to promote the real-world behavior of the user.

Embodiments also include a simulated reality platform for rehabilitating users of the platform by promoting real-world behaviors of users engaged with simulations. The platform can include a cloud subsystem that can create and store a library of simulations each including a set of scenes configured to promote real-world behaviors of users engaged with simulations. A client subsystem can administer a particular simulation provided by the cloud subsystem for a particular user such that the user experiences a course of a subset of a set of scenes. Lastly, a user subsystem includes a head mounted near-to-eye display operable to render the subset of scenes as administered by the client subsystem to promote a real-world behavior of the user engaged in the simulation.

Embodiments include a method performed by a user computer of a simulated reality platform for simulating real-world scenes to promote a real-world behavior of a user immersed in a simulation. The method can include initiating a session for a real-world simulation including a real-world scene selected from many real-world scenes of the real-world simulation, receiving an authentication code to enable the session for the user of the real-world simulation to experience the selected real-world scene, and upon successful authentication of the user based on the authentication code, launching the session to render the real-world simulation including the selected scene for the user as authorized by the authentication code.

Embodiments include a head mounted display (HMD) system including a chassis, and one or more displays mounted to the chassis, to render a scene of a simulated reality for an optical receptor of a user when the HMD system is worn by the user. The simulation can include a number of scenes that can promote a real-world behavior by the user wearing the HMD system. The HMD can also include a camera mounted to the chassis, to capture movement of the optical receptor responsive to the scenes. The HMD system can also include a network interface that can communicate with a client subsystem configured to administer the simulation.

Embodiments also include a method performed by a client computer administering a simulated reality by a user device to promote a real-world behavior of a user engaged with a simulation. The method includes connecting the client subsystem to a cloud service by calling an application programming interface (API) of the cloud service to grant access to the simulated reality platform such that the client computer administers a session including a simulation of a real-world scene configured to promote a real-world behavior of the user engaged with the simulation, and causing the user computer to render the simulation of the real-world scene under control of the client computer and in accordance with an authorization granted by the cloud service.

Embodiments include a method performed by one or more server computers of a simulated reality platform for administering a simulation to promote a real-world behavior of a user engaged with the simulation. The method can include creating simulations that can promote real-world behaviors by users, where each simulation includes scenes. The method can further include creating a user profile including information indicating an ailment of the user for which the user seeks rehabilitation, identifying one or more simulations including scenes to promote the desired real-world behavior to rehabilitate the user, linking the user profile to the one or more identified simulations, and enabling the simulation capable of rehabilitating the user, the simulation including a course for traversing through a subset of the scenes. Lastly, the method can include adjusting the course to traverse a different subset of the scenes in response to the user failing to demonstrate the desired real-world behavior.

### BRIEF DESCRIPTION OF THE DRAWINGS

One or more embodiments of the present invention are illustrated by way of example and not limitation in the figures of the accompanying drawings, in which like references indicate similar elements.

FIG. 1 illustrates a user engaged with components of a simulated reality rehabilitation system according to some embodiments of the present disclosure;

FIG. 2 illustrates an example of a scene of a rehabilitation session according to some embodiments of the present disclosure;

FIG. 3 is a block diagram illustrating a cloud stack and a client stack of a simulated reality rehabilitation system collectively operable to administer a session by a near-to-eye display system according to some embodiments of the present disclosure;

FIG. 4 is a block diagram of a stack for managing multiple simulation sessions according to some embodiments of the present disclosure;

FIG. 5 illustrates an experience flow or logical diagram for a rehabilitation session according to some embodiments of the present disclosure;

FIG. 6 is a block diagram illustrating an simulated reality rehabilitation system and processes performed therewith according to some embodiments of the present disclosure;

FIG. 7 is a flowchart illustrating a process performed by a server computer of the simulated reality rehabilitation platform according to some embodiments of the present disclosure; and

FIG. 8 is a block diagram illustrating a computer device configured to implement aspects of the disclosed technology according to some embodiments of the present disclosure.

### DETAILED DESCRIPTION

The embodiments set forth below represent the necessary information to enable those skilled in the art to practice the embodiments, and illustrate the best mode of practicing the embodiments. Upon reading the following description in light of the accompanying figures, those skilled in the art will understand the concepts of the disclosure and will recognize applications of these concepts that are not particularly addressed herein. It should be understood that these concepts and applications fall within the scope of the disclosure and the accompanying claims.

The purpose of terminology used here is only for describing embodiments and is not intended to limit the scope of the disclosure. Where context permits, words using the singular or plural form may also include the plural or singular form, respectively.

As used herein, unless specifically stated otherwise, terms such as “processing,” “computing,” “calculating,” “determining,” “displaying,” “generating” or the like, refer to actions and processes of a computer or similar electronic computing device that manipulates and transforms data represented as physical (electronic) quantities within the computer's memory or registers into other data similarly represented as physical quantities within the computer's memory, registers, or other such storage medium, transmission, or display devices.

As used herein, the terms “connected,” “coupled,” or variants thereof, refer to any connection or coupling, either direct or indirect, between two or more elements. The coupling or connection between the elements can be physical, logical, or a combination thereof.

The disclosed technology leverages advancements in virtual and augmented reality technology to prevent and treat patients with substance use disorders along with rehabilitating repeat offenders. The disclosed technology is a powerful technique to rehabilitate rather than just punish individuals. The scope of this disclosure includes formal education, vocational training, psychological rehabilitation, and correctional services rehabilitation. The disclosed technology includes services in a telemedicine context and can extend to hospitals, rehab centers, correctional officers, inmates, etc.

The disclosed technologies include virtual reality platforms for corrections applications. For example, these products create a virtual reality environment to help rehabilitate inmates by exposing them to “scenarios” similar to the real-world and provides guidance for responding to those scenarios. In other words, users are exposed to real-world scenarios that would trigger users to relapse, but are trained in a simulated world to respond appropriately.

The disclosed embodiments further expand on these technologies by similarly including a simulated reality environment that helps rehabilitate inmates (or more broadly anyone that could benefit from rehabilitation). Some embodiments include an expert component that collects data (e.g., from the simulated reality environment, physiological data) and applies machine learning to the data in order to assess the user, predict future user behavior, and identify suitable treatments. For example, an outcome of this process may be used to determine that a user exposed to a virtual reality environment is not suitable for parole or needs therapy to help succeed in the real-world.

The disclosed technology can reduce recidivism and relapse rates through virtual reality or augmented reality based immersive learning and rehabilitation programs. In particular, the immersive technology is a solution that can allow users to undergo correctional rehabilitation services for sex offenses, family violence, alcoholism, as well as other offenses. The disclosed solution can also assist in treating patients' psychological problems including mental illness, emotional disorders, co-existing disorder, intermittent explosive disorder, and others. The disclosed immersive technology also has a broad range of other applications. Examples include immersive formal education programs that are used to strengthen the mastery of the English language, business, mathematics, sciences, technology, along with other curriculum. Users can also acquire new vocational training skills such as car mechanic, plumbing, welding, carpentry, along with other professions.

The disclosed technology can be implemented in a telemedicine context. For example, a simulated reality rehabilitation system can include the use of use of telecommunication and information technology to provide rehabilitation services from a distance. This can been used to overcome distance barriers and to improve access to rehabilitation services that are often not consistently available in distant communities.

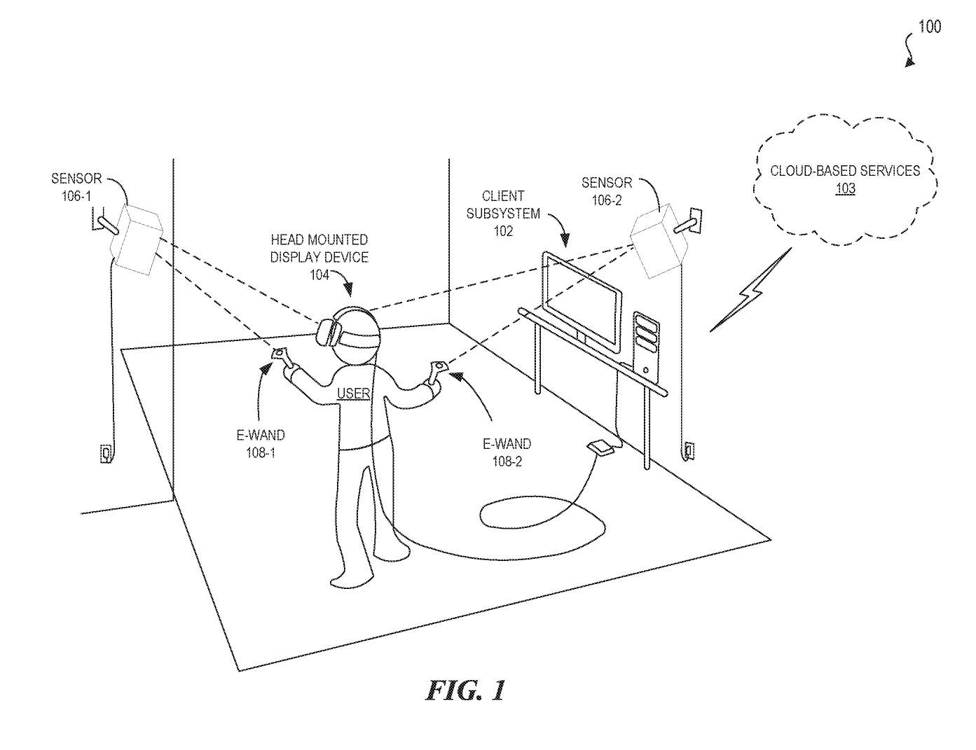

FIG. 1 illustrates a user engaged with components of a simulated reality rehabilitation system (the “system”) according to some embodiments of the present disclosure. The components 100 can include a client computer subsystem 102 that administers a simulation session running on components of a user computer subsystem including a head mounted display (HMD) device 104, motion or position sensors 106, electronic wands 108, etc. In some embodiments, some of the components 100 are remotely located from the user. For example, cloud components can provide cloud-based services 103 to administer the simulation session running on the components of the user computer subsystem or provide services or content for the client subsystem 102 to administer simulation sessions. Hence, administration of a simulation session could be on the HMD device 104 or a remote system that receives session progress feedback (e.g., anywhere outside of room where the user is experiencing a simulation).

As shown, the client subsystem 102 includes a desktop computer that can provide content of an simulation session to the components of a user subsystem and process feedback from the user subsystem. As shown, the HMD device 104 is a near-to-eye display system that is worn by a user. For example, the HMD device 104 can have a chassis and various electrical and optical components to enable an immersive experience by the user wearing the HMD device 104. For example, the HMD device 104 can include a display for each of the user's eyes. The displays can render a real-world scene of a simulation for view by the user's eyes when the HMD device 104 is worn by the user. The HMD device 104 may also include a camera mounted to the chassis. The camera may capture movement of the user's pupils for physiological feedback responsive to simulated real-world scenes being rendered. The HMD device 104 may also include a network interface enabling the client subsystem 102 to communicatively couple the user subsystem to the client subsystem over a wired or wireless connection.

In some embodiments, the HMD device 104 could include features for measuring the user's physiological activity. For example, the HMD device 104 may include components to measure the user's electrical brain activity. As such, the HMD device 104 can also collect physiological data in combination with any direct input by the user. In some embodiments, the physiological data can be used to supplement the user's conscious inputs. In some embodiments, the physiological data could be used to compare against the user's conscious input to detect when the user is attempting to deliberately fool the system. In this way, the system can detect the user's true progress in a rehabilitation program in real-time and adjust a course accordingly.

The HMD device 104 can be used for rendering a virtual immersive environment by displaying images in view of the user's eyes such that the user can only see the images and see nothing in the real world. The HMD device 104 can also render an augmented immersive environment. As such, the user can still see the real world even while the HMD device 104 is worn by the user. To achieve an augmented reality, the user in an augmented reality simulation has a transparent view with digital objects overlaid or superimposed on the user's real-world view.

Examples of other components of the user subsystem include the sensors 106, which could include cameras or motion detectors that are positioned proximate to the user such that the sensors 106 can obtain real-world feedback responsive to interactions with a simulated real-world scene. For example, cameras facing the user can detect the user's movement while the user is engaged in a simulation and provide feedback to the client subsystem 102 administering the simulation. Other components include means for the user to consciously input answers to questions about a rehabilitation course. Examples of input devices include the handheld electronic wands 108 (“e-wands 108”), which can include buttons for the user to input data and/or accelerometers that detect spatial movement. For example, the user can move the wands 108 to provide inputs responsive to a scene administered by the client subsystem 102.

A simulation session can include one or more scenes or scenarios that each simulate a real-world experience. For example, a scene could simulate a person offering the user to buy or use drugs. Another scene could simulate an interaction between the user and the user's spouse or partner after the offer to use or buy drugs. Depending on the interaction between the user and the spouse or partner, the simulation may continue to one of multiple alternative scenes. One scene could simulate an interaction between the user and a police officer after the interaction with the spouse or partner. An alternative scene may be of a follow-up interaction with the person who offered the user drugs.

A rehabilitation program includes any number of real-world scenes and/or levels of scenes that collectively form a simulation. The scenes have content and can be rendered in different orders depending on the interaction with a particular user. As such, the rehabilitation program can be personalized for different users. The particular scenes and the order in which they are rendered constitute a course for the rehabilitation program. A user can be assigned a course at the beginning of a rehabilitation program, and the course can change while the program is running in response to how the user is interacting with rendered scenes. For example, a service administering a simulation session could dynamically change to repeat a similar scene if the user failed to successfully show progress in a current scene.

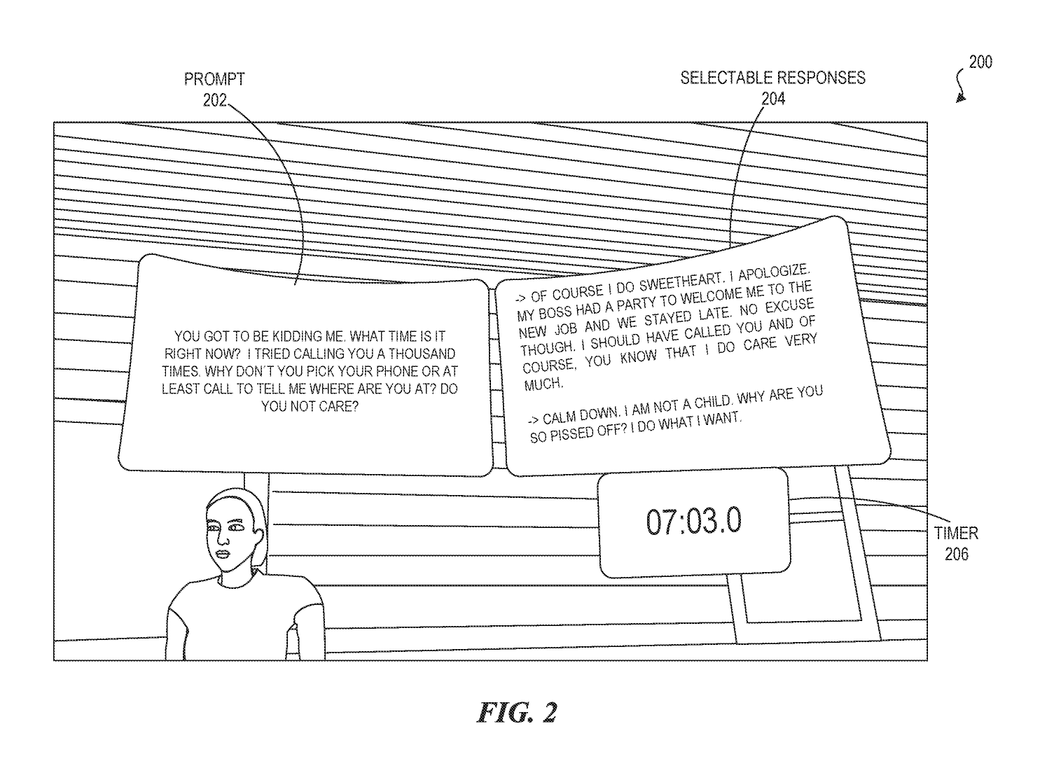

FIG. 2 illustrates an example of a real-world scene 200 of an rehabilitation session according to some embodiments of the present disclosure. The scene 200 depicts a virtual person attempting to interact with the real user of the simulation. The virtual person prompts the user to respond to a question 202. The user is presented with alternatively selectable responses 204 to the question 202. The user can then select one of two of the responses 204. The selected response can be used to determine whether the user is progressing successfully through the rehabilitation program. If the user successfully completed the scene 200, the user may progress through a remaining course of scenes. On the other hand, if the user failed to successfully complete the scene 200, the course of scenes may be changed to adapt to the user's lack of progress. As shown, the scene 200 also depicts a timer 206 that counts down to encourage the user to promptly respond.

The simulated reality rehabilitation system can evaluate other factors to determine a suitable course for a user engaged in a rehabilitation scene. For example, an HMD can include sensors that measure the physiological responses of a user such as heart rate or eye movement. These measurements are received as inputs and can be used as metadata associated with a scene and/or the user's selected response or other input. For example, the system can detect how a user responds to a scene that depicts a narcotic substance. This metadata can be used to assess whether the user's physiological responses indicate successful rehabilitation or even whether the user's selected responses are inconsistent with the user's physiological activity. In other words, measuring physiological factors allows the system to determine whether the user is attempting to deceive the system.

In some embodiments, the system can include a library of rehabilitation programs. As described further below, the system can include servers that are remotely located from client systems that can access a rehabilitation program administered to a user system (e.g., an HMD). In some examples, a particular client system can access a particular rehabilitation program and a particular course of scenes that is personalized for a particular user. The particular client system can administer the particular program that runs by a user system as administered by the client system.

Further, a local software generation and distribution framework can be used to rapidly scale content. The core essential components and services can support complex user, curriculum, and session elements that can be easily managed by a service provider. As such, a platform of the simulated reality rehabilitation system can standardize interaction elements such as a session landing, sign-in, navigation rules, and the like. A top level abstraction layer can support customization such as a sequence of sessions or scenes or conditional ordering of sessions or scenes. Services can include authentication, tracking, reports, user services, help services, pause and resume services, and the like.

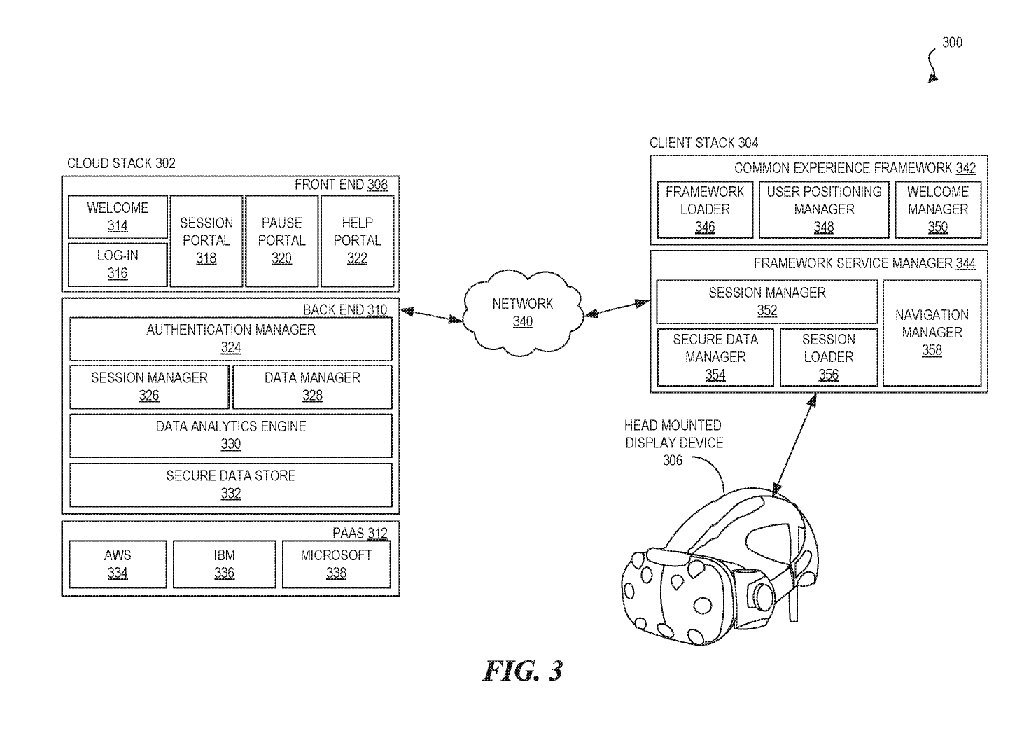

For example, FIG. 3 is a block diagram illustrating a cloud stack 302 and a client stack 304 of a simulated reality rehabilitation platform 300 (“platform 300”) of a simulated reality rehabilitation system collectively operable to administer a simulation session on a head mounted display (HMD) device 306 (or, more generically, a near-to-eye display system) according to some embodiments of the present disclosure.

As shown, the cloud stack 302 includes three primary layers: a front end layer 308, a back end layer 310, and a platform as a service (PaaS) layer 312. The front end layer 308 includes a welcome component 314 and a log-in component 316. The two components 314 and 316 are executed at the beginning of a rehabilitation program administered to orient a user and seek login credentials to control access to rehabilitation programs and user information of the platform 300. The front end layer 308 also include a session portal 318, pause portal 320, and help portal 322. The session portal 318 is for normal front facing operations of a simulation session whereas the pause portal 320 is for operations while the session is paused. Lastly, the help portal 322 is for helping the user or administrator to address questions related to the platform 300 or simulation.

The back end layer 310 includes an authentication manager 324 that can authenticate a user and/or an administrator of the platform 300. A session manager 326 can manage access to a particular session. A data manager 328 can manage user data and/or data about the session such as any feedback from users while engaged in sessions. For example, the data manager 328 can collect feedback data from users including their conscious inputs and physiological data. A data analytics engine 330 can process the collected data to determine the progress of users and to learn how to improve the rehabilitation programs (e.g., sessions, courses, scenes). A secure data store 332 can store sensitive data such as data that identifies users or their ailments. Lastly, the PaaS layer 312 includes cloud computing services that provide the platform 300 for clients to administer the simulation sessions. Examples include AMAZON WEB SERVICES (AWS) 334, or services provided by IBM 336 and/or MICROSOFT 338.

The cloud stack 302 is communicatively connected to the client stack 304 over a network 340 such as the internet. The client stack 304 includes a common experience framework layer 342 and a framework service manager layer 344. The common experience framework layer 342 includes a framework loader 346 to load the framework for a session, a user positioning manager 348 to monitor and track the relative position of the user engaged with the session, and a welcome manager 350 to orient the user at the beginning of the session.

The framework service manager layer 344 includes a session manager 352 to manage the session experienced by a user wearing the HMD device 306. The framework service manager layer 344 also includes a secure data manager 354 to store or anonymize any sensitive data (e.g., identifying users or their ailments), session load manager 356 for loading a session, and a navigation manager 358 for navigating a user through a course of scenes of a rehabilitation program. The platform 300 is merely illustrative to aid the reader in understanding an embodiment. Other embodiments may include fewer or additional layers/components known to persons skilled in the art but omitted for brevity.

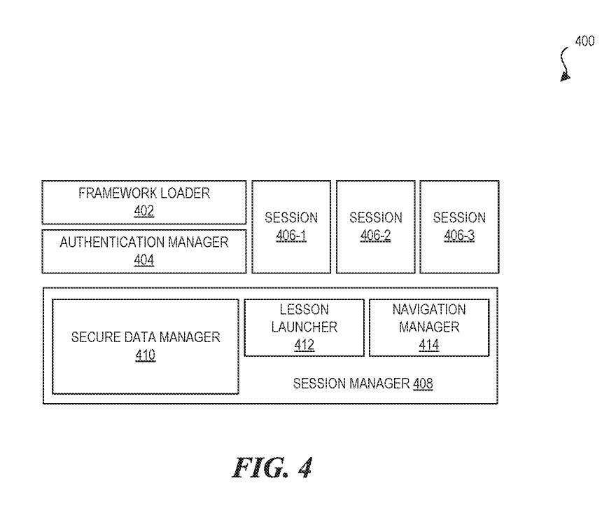

For example, FIG. 4 is a block diagram of a stack 400 for managing multiple simulation sessions. As shown, the top layer includes a framework loader 402 and authentication manager 404 to load applications and authenticate users and/or administrators of the simulated reality rehabilitation system. Multiple sessions 406 can be executed as part of the framework. A session manager 408 includes a secured data manager 410 for securing any sensitive data, a lesson launcher 412, and a navigation manager 414. As such, the session manager 408 can harmonize the various elements of multiple sessions 406.

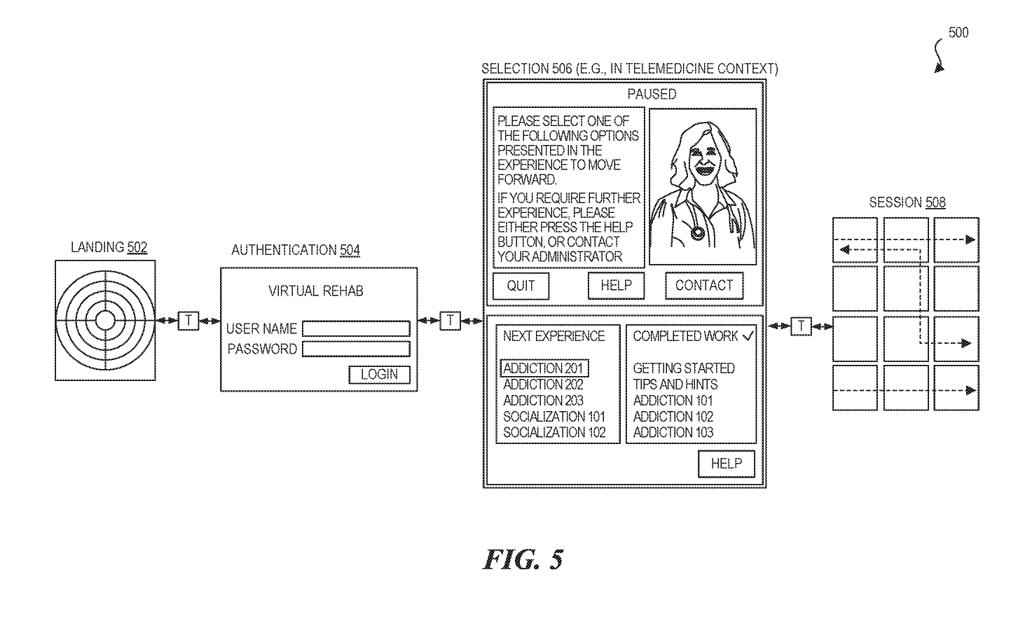

FIG. 5 illustrates an experience flow or logical diagram 500 for a rehabilitation session according to some embodiments of the present disclosure. In the landing experience 502, a number of preliminary actions are taken when a user initially engages with the simulated reality rehabilitation system (“system”). In some embodiments, the landing experience 502 can create a smooth transition into a virtual space with open space and soothing music. For example, it can render written or audible greetings, advertisements, or prompts to nudge a user via cues to the middle of a virtual space by moving in real-world space.

This includes loading a number of features such as a positioning system. The relative position of a user in a physical room can be determined with a number of sensors in different locations of the room. The sensors can be part of a positioning system that executes time-of-flight techniques that measure the time that it takes for a signal to travel to an object and return back to cameras. The measurements can be used to determine the relative distance from the sensors. Further, a number of distances determined for a number of sensors can be used to triangulate the position of the user. In some instances, the landing experience can also include a branding feature to display a brand of the company administering the rehabilitation program.

In some embodiments, the user can automatically transition (denoted with a “T” in FIG. 5) to an authentication experience 504 once the user is in the middle of the landing room. The authentication experience 504 may present a user with a log-in screen. Upon successful login, is check can be performed to see if the user has run the tutorial. If not, the user can be offered to view the tutorial. If the user fails to enter the correct password after a certain number of times, the system may automatically exit, and the facilitator of the session may be notified of the failed attempt to long in.

The authentication experience 504 includes features to secure access to a rehabilitation program and sensitive user data such as data identifying users or their ailments. In some embodiments, the authentication experience 504 can prompt a user for a user name and password that is used to authenticate the user. The authentication experience 504 may include a “welcome room” including tutorials or features to orient the user about how to engage with a rehabilitation program. For example, a user orientation can explain the various controls used to input responses or give a guided tour through a program, course, or scenes. In this way, the user can prepare to effectively utilize a rehabilitation program.

Once authenticated, the user can transition to a selection experience 506 to engage in a simulation session. For example, after a successful login, the user may be asked to click a virtual transition control. Once the transition control is clicked, the user transitions to a selection room of the selection experience 506. The selection experience 506 can present an overview of a session including a course of scenes for the user to experience. The overview can include a description of options including selectable scenes, quit, help, and contact controls. In some embodiments, selecting a contact or help control will open a chat or video conference with an administrator or professional. As shown, the selection experience 506 allows the user to select scenes to experience, and presents a tracking of completed work of a rehabilitation program. In some embodiments, the selection portal can also include options for reporting results. In some embodiments, the selection experience 506 can be presented to a user to give that user control over his or her own experience.

In some embodiments, the disclosed technology can be implemented in a telemedicine context. For example, a simulated reality rehabilitation system can include the use of telecommunication and information technology to provide rehabilitation services remotely. For example, a scene selection portal can be presented to an administrator or professional rather than the user. Hence, the administrator can select scenes for the user and monitor the user's progress. In some embodiments, the selection portal is presented at the beginning before a session commences and/or when a session is paused. This enables changing a course of scenes at any point during a session.

The user can then transition to a simulation of a session experience 508. Each block represents a scene including simulated real-world content. The scenes can be arranged as layers and could be ordered to achieve certain rehabilitation goals. For example, each row could represent an ordered set of scenes of a particular experience including different content meant to progress a user through a rehabilitation exercise. Further, each column could represent scenes with similar objectives but including different content.

As shown, each broken arrow depicts examples of courses that a user can traverse through a number of scenes in a session. In particular, three broken arrows are illustrated as three paths through different scenes. The uppermost broken arrow represents a course that traverses through three scenes in a single direction. The lowermost course traverses through three different scenes in the same direction. The uppermost and lowermost courses could represent similar rehabilitation exercises that use different content. The remaining course traverses through five scenes in different directions. As such, a user can traverse through similar scenes that use different content or repeat scenes if the user fails to succeed in a scene.

Hence, the system can use a highly structured and formalized navigation paradigm. By highly structuring the navigation through scenes, the system is scalable to engage multiple users experiencing multiple sessions. In other words, high scalability is based on formalization of common elements of that are common to all experiences. For example, the navigation paradigm can include designs for experiences around a “room centered” model (e.g., a walk around experience). The navigation paradigm can use navigation cues to reduce confusion and exploration outside of an intended experience. The navigation paradigm may identify generic feedback prompts such as text, voice, haptic feedback, sounds, etc. In some embodiments, the navigation paradigm can define all transitions from room-to-room in a simple consistent manner. Thus, the navigation paradigm can include a navigation map builder that controls session flows.

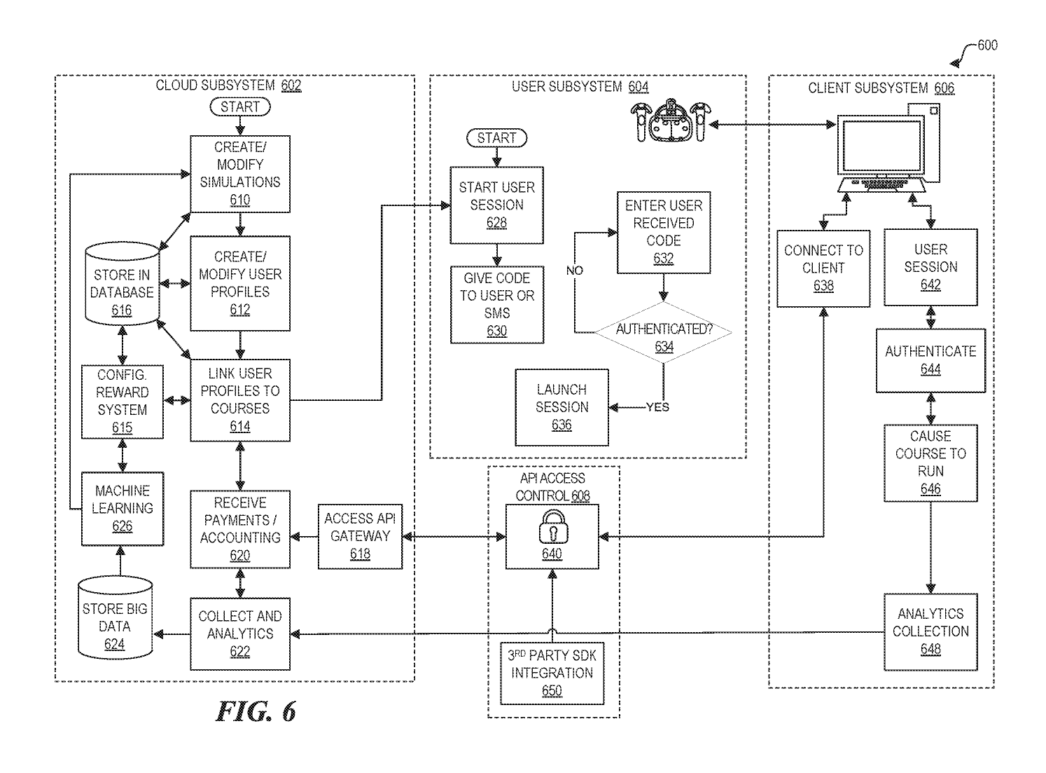

FIG. 6 is a block diagram illustrating a simulated reality rehabilitation System 600 (“system 600) and processes executed therewith according to some embodiments of the present disclosure. As shown, the system 600 includes a cloud subsystem 602, a user subsystem 604, and a client subsystem 606. As shown, the system 600 also includes an application programming interface (API) access control component 608 that secures communications between subsystems of the system 600.

The system 600 can collectively form a simulated reality platform for rehabilitating users of the platform by promoting real-world behaviors of users engaged in simulations. The cloud subsystem 602 can create and store a library of simulations each including one or more scenes that promote real-world behaviors of users engaged with simulations. The client subsystem 606 can administer a particular simulation provided by the cloud subsystem 602 for a particular user such that the user experiences a course of at least some scenes. The user subsystem 604 can include a head mounted near-to-eye display that can render administered by the client subsystem 606 to promote a real-world behavior of a user engaged in the simulation.

The networks interconnecting the system 600 may include any combination of private, public, wired, or wireless portions. The data communicated over the networks may be encrypted or unencrypted at various locations or along different portions of the networks. Each component of the system 600 may include combinations of hardware and/or software to process data, perform functions, communicate over the networks, and the like. For example, a component of the system 600 may include a processor, memory or storage, a network transceiver, a display, an operating system and application software (e.g., for providing a user interface), and the like. Other components, hardware, and/or software included in the system 600 that are well known to persons skilled in the art are not shown or discussed herein for brevity.

The system 600 can include different computing devices. For example, the client subsystem 606 may include a server or other devices to interact with the system 600 or serve other devices the system 600. Examples of these devices include smart phones (e.g., APPLE IPHONE, SAMSUNG GALAXY, NOKIA LUMINA), tablet computers (e.g., APPLE IPAD, SAMSUNG NOTE, AMAZON FIRE, MICROSOFT SURFACE), computers (e.g., APPLE MACBOOK, LENOVO 440), and any other device that can couple to the system 600.

The cloud subsystem 602 can execute processes for rehabilitating users of the system 600. For example, one or more servers can facilitate creating a number of simulations to promote real-world behaviors of users engaged in the simulations. For example, in block 610, the cloud subsystem 602 can create or modify a number of simulations that can include a combination of ordered or unordered real-world scenes. This may include a number of courses traversing the scenes that can adapt as needed to rehabilitate users. In some embodiments, the arrangement of scenes and the courses traversing the scenes can be stored as a navigation map for use in real-time during the execution of a simulation to dynamically adjust users' experiences in an effort to optimize the effect of promoting desired real-world behaviors of users.

In block 612, the cloud subsystem 602 can create or modify a number of user profiles. A user profile can include data that identifies a user, health-related information, access rights granted to that user, as well as ailments (e.g., an addiction) for which the user is seeking rehabilitation. As part of the rehabilitation, the cloud subsystem 602 can identify simulations that would promote desired real-world behaviors to rehabilitate the user.

In block 614, the cloud subsystem 602 can formulate a navigation map for the user to undergo a rehabilitation treatment by experiencing a simulation of scenes, and store the navigation map in the user profile. Thus, a navigation map can link a user to one or more simulations to rehabilitate the user, and that linkage can be stored in a database.

In some embodiments, the cloud subsystem 602 includes a reward system. For example, in block 615, a reward system is configured to reward a user for correcting or improving his or her behavior to achieve a desired behavior. In particular, a user can be rewarded in response to demonstrating a corrected or improved behavior. The system 600 may decide between different rewards responsive to the user's corrected or improved behavior. Examples of rewards include navigation options and/or redeemable points. For example, a user may be allowed to advance to a next scene or level, move between scenes or levels, or skip scenes or levels of a rehabilitation program. In some embodiments, the user is awarded redeemable points for passing a scene or level. The points may be redeemed for access to real-world items such as being granted access to a library or computing resources for a period of time. The data associated with the reward system may be stored in the database. For example, the rewards data may be stored with the user's profile.

In some embodiments, the cloud subsystem 602 can receive payments to access rehabilitation services. For example, the cloud subsystem 602 may provide rehabilitation services that can be purchased per session, course, or the like. For example, in block 618, the client subsystem 606 can access an API gateway by making a payment as in block 620 for a service from the cloud subsystem 602. Thus, the cloud subsystem 602 can enable a simulation capable of rehabilitating the user, where the simulation includes a course for traversing through a subset of scenes.

The cloud subsystem 602 includes feedback loops to adjust a course in real-time and adjust the simulations, courses, and scenes based on machine learning. For example, the cloud subsystem 602 can adjust a course to traverse a different subset of scenes in response to a user failing to demonstrate the desired real-world behavior.

In block 622, the cloud subsystem can collect feedback data from one or more client subsystems of users engaged in various simulations as part of rehabilitation treatments. The collected data can be analyzed to determine the effectiveness of the simulations to rehabilitate users.

In block 624, the raw collected data and/or the results of any analysis of that data can be stored for subsequent use in a machine learning process of block 626. The results of the machine learning process of block 626 can be used to update the simulations subsequently used to rehabilitate users.

The user subsystem 604 of a simulated reality platform can include an HMD device having one or more displays mounted to a chassis of the HMD. The displays can render scenes of a simulated reality for the user's eyes when the user is wearing the HMD device. The simulation can include scenes configured to promote a real-world behavior by the user wearing the HMD device. The HMD device can include cameras mounted to its chassis. The cameras can capture movement of the user's eyes as feedback to the simulated scenes. The HMD device can also include a network interface to communicate with a client subsystem and/or the cloud subsystem 602 over one or more networks.

In some embodiments, the HMD device can create an augmented reality experience by rendering a scene on the displays to overlay a view of a real-world environment on the user's eyes. In some embodiments, a scene is rendered on the displays to create a virtual reality view by the user's eyes.

The user subsystem 604 may include other computing devices used for a rehabilitation simulation. For example, the user subsystem 604 may include sensors that can detect the user's position or movement while the user is engaged in a simulation and provide feedback to the client subsystem 606 administering the simulation for that user. Examples of other computing devices include handheld electronic wands that can receive input from a user engaged in a simulation based on the spatial movements of the electronic wands.

The user subsystem 604 can perform various processes of the simulated reality platform for simulating real-world scenes to promote a real-world behavior of a user immersed in a simulation. In block 628, a real-world simulation session is initiated. The session includes a real-world scene selected from multiple real-world scenes of simulations. In response to initiating a session, the cloud subsystem can link the user profile to the session.

In block 630, a code is given to the user to grant access to the requested simulation. The code can be given to the user over a communications channel other than the channel used to link the user profile to the session. For example, the code can be sent to the user's smartphone in an SMS text message. In some embodiments, the code is a multi-digit passcode.

In block 632, the user enters the code to access the desired session. In some embodiments, the code enables the session for the user of the simulation to experience the selected real-world scene. Hence, in block 636, upon successful authentication in block 634 based on the authentication code, the session is launched to render the real-world simulation including the selected scene for the user as authorized by the authentication code. In block 634, if the code is not authenticated, the user is requested to re-enter a code.

In some embodiments, the code authenticates the user, the session, the selected scene, or combinations thereof. For example, the code may authenticate the user and the session but not a scene selected by the user. In some embodiments, the scene can be selected by different parties. For example, the scene can be selected autonomously without intervention by the user (e.g., based on the user's profile alone) or by an administrator such as a healthcare professional specializing in rehabilitation, in a telemedicine context. In another example, the scenes of the simulation is a sequence of ordered scenes and the selected scene is authenticated only if any prior scenes of the sequence have been successfully completed by the user.

The client subsystem 606 can perform various processes for simulating real-world scenes to promote a real-world behavior of a user immersed in a simulation. In block 638, the client subsystem 606 can connect with the cloud subsystem 602 by calling an API in block 640 to gain access to the simulated reality platform.

In block 642, the client subsystem 606 can commence the user's session including a simulation of a real-world scene configured to promote a real-world behavior of the user engaged with the simulation. In some instances, the client subsystem 606 may not be authorized by default to administer certain simulations, scenes, or courses of scenes. Hence, the client subsystem 606 may need to authenticate itself to continue the simulation.

In block 644, the client subsystem 606 can be authenticated to cause a particular session, scene, or course to run using the user subsystem 604. For example, in block 616, the client subsystem 606 may authenticate a course for the user subsystem 604 to render a simulation. The client subsystem 606 can cause a selected course to run for a particular simulation or select a different course for the simulation. In some embodiments, the scene is included in a sequence of ordered real-world scenes for the session. Thus, the client subsystem 606 can cause the user subsystem 604 to render a simulation of the real-world scene under the control of the client subsystem and in accordance with an authorization granted by the cloud subsystem 602.

In block 648, the client subsystem 606 can collect data of the simulation in real-time. The data may indicate inputs by the user of the client computer in response to the simulation. The client subsystem 606 can perform various analytics such as determining whether the user is being successfully rehabilitated based on the collected data in block 648.

Lastly, the system 600 can enable third party software development kit (SDK) integration in block 650. In particular, the API access control 608 includes an interface 640 for the third party SDK to connect to the cloud subsystem 602 or client subsystem 606. As such, a third party can participate in the simulation for rehabilitating users.

Although FIG. 6 includes a sets of ordered blocks, the disclosure is not so limited. For example, any of the blocks of FIG. 6 can occur in another order. Some embodiments may include fewer blocks or additional blocks that would be known to persons skilled in the art in light of the disclosure.

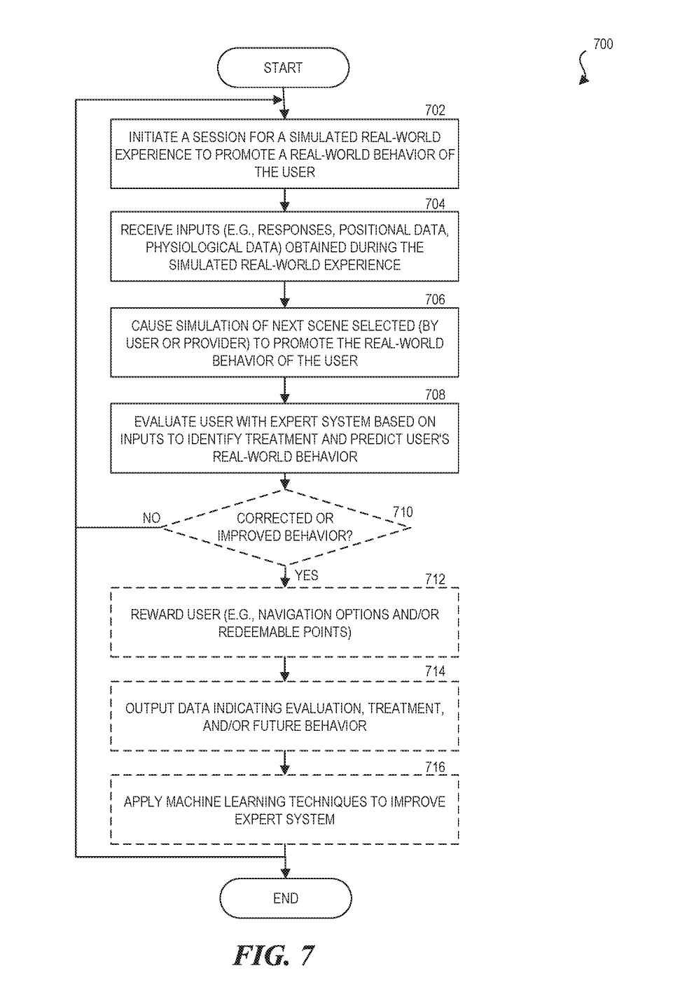

FIG. 7 is a flowchart illustrating a method 700 performed by the simulated reality platform according to some embodiments of the present disclosure. More specifically, the method 700 can be performed by a server computer system (e.g., a cloud-based computer system) of the simulated reality platform for rehabilitating users of the platform by promoting real-world behaviors of users engaged with simulations.

In step 702, the server computer system initiates a session for a simulated real-world experience. In some embodiments, the simulated real-world experience can be an augmented or virtual reality simulation. The simulated real-world experience includes a real-world scene selected from multiple available real-world scenes. In some embodiments, a real-world scene is selected by a third-party provider or a user engaged with the simulated real-world experience. The simulated real-world experience can promote a real-world behavior of a user engaged with the simulated real-world experience. For example, the real-world behavior can mitigate a risk of recidivism.

In step 704, the server computer system can receive inputs obtained during the simulated real-world experience. The received inputs can include user responses to prompts, real-world positional data of the user, and/or real-world physiological data of the user. For example, each scene can elicit an alternatively selectable response from the user. The real-world physiological data can include, for example, blood pressure data, brainwave activity data, eye movement data, and/or heart electrical activity data. In some embodiments, the received inputs may be anonymized to remove data identifying the user.

In step 706, the server computer can cause a next real-world scene to render in the simulated real-world experience. The next real-world scene is selected based on at least some of the received inputs. In some embodiments, scenes elicit the user to select one of many responses, and next scenes increase in complexity with successful selection of responses to prior scenes. For example, a more complex scene can have a greater number of selectable responses compared to a less complex scene. In some embodiments, determining the next scene involves identifying a scene of a different scene as that next scene of the selected scene. In some embodiments, the scenes are rendered seamlessly to promote the real-world behavior.

In step 708, the user is evaluated based on the received inputs with an expert system to predict the user's real-world behavior and identify a corresponding treatment. The expert system may implement artificial intelligence to evaluate the user. The expert system may be a cognitive behavioral system for rehabilitating the user. Further, evaluating the user may involve comparing conscious user inputs to real-world physiological data to determine whether, for example, the user is attempting to deceive the simulated real-world experience. In some embodiments, evaluating a user is based on a time interval between two inputs or between a user's reaction to a stimuli, which can be purposefully included in scenes to trigger a reaction by the user.

In step 710, the server computer can determine whether the user has shown a correction or improvement in behavior towards achieving the desired real-world behavior. The user may be rewarded if a correction or improvement of the user's behavior has been determined. On the other hand, if the user's behavior has not corrected or improved, the rehabilitation program may iterate through one or more scenes to promote a desired correction or improvement.

In step 712, the user is rewarded in response to corrected or improved behavior. The server computer system may decide between different rewards for the user in response to the determination that the user corrected or improved his or her behavior. Examples of rewards include navigation options and/or redeemable points. For example, a user may be allowed to advance to a next level, move between levels or skip levels of a rehabilitation program. In some embodiments, the user is awarded redeemable points for passing a level. The points may be redeemed for access to real-world items such as being granted access to library hours or computing resources.

In step 714, the server computer can output data indicative of the evaluation, the predicted user's real-world behavior, and/or the treatment. In some embodiments, the treatment is a recommended therapeutic treatment identified from multiple therapeutic treatments. In some embodiments, the output includes recommending the treatment to a third-party provider of the simulation.

In step 716, the server computer executes machine learning based on the received inputs to improve the expert system for simulated real-world experiences that promote real-world behaviors and improve predicting real-world behaviors or identifying treatments. In some embodiments, the machine learning is supervised by a third-party provider of the simulation or is unsupervised. In some embodiments, the server computer system can improve selection of a next scene for a scene to promote a real-world behavior of a user of the simulation in accordance with the machine learning.

Although FIG. 7 illustrates a particular set of ordered steps, the disclosure is not so limited. For example, any of the steps 702 through 716 can be practiced in another order. Some embodiments may include fewer steps or additional steps that would be known to persons skilled in the art in light of the disclosure.

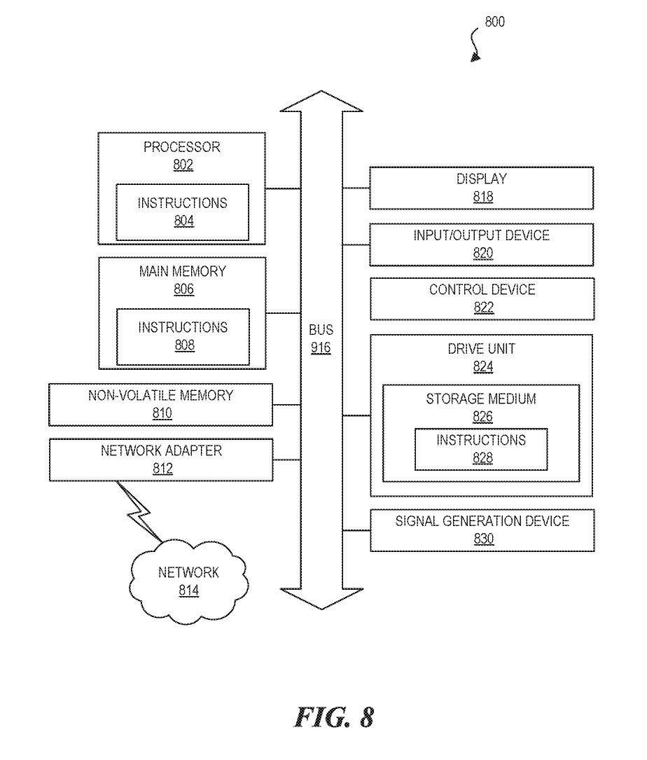

FIG. 8 is a block diagram illustrating an example of a computing system 800 in which at least some operations described herein can be implemented. For example, the computing system 800 may be responsible for sampling or collecting data related to data. The computing system 800 may include one or more central processing units (e.g., processors 802), main memory 806, non-volatile memory device 810, network adapter 812 (e.g., network interfaces), display 818, input/output devices 820, control device 822 (e.g., keyboard and pointing devices), drive unit 824 including a storage medium 826, and signal generation device 830 that are communicatively connected to a bus 816.

The bus 816 is illustrated as an abstraction that represents any one or more separate physical buses, point to point connections, or both connected by appropriate bridges, adapters, or controllers. The bus 816, therefore, can include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire.” A bus may also be responsible for relaying data packets (e.g., via full or half duplex wires) between components of a network appliance, such as a switching engine, network port(s), tool port(s), etc.

In some embodiments, the computing system 800 operates as a standalone device, although the computing system 800 may be connected (e.g., wired or wirelessly) to other machines. For example, the computing system 800 may include a terminal that is coupled directly to a network appliance. As another example, the computing system 800 may be wirelessly coupled to the network appliance.

In various embodiments, the computing system 800 may be a server computer, a client computer, a personal computer (PC), a user device, a tablet PC, a laptop computer, a personal digital assistant (PDA), a cellular telephone, an iPhone, an iPad, a Blackberry, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by the computing system.

While the main memory 806, non-volatile memory 810, and storage medium 826 (also called a “machine-readable medium) are shown to be a single medium, the term “machine-readable medium” and “storage medium” should be taken to include a single medium or multiple media (e.g., a centralized or distributed database, and/or associated caches and servers) that store one or more sets of instructions 828. The term “machine-readable medium” and “storage medium” shall also be taken to include any medium that is capable of storing, encoding, or carrying a set of instructions for execution by the computing system and that cause the computing system to perform any one or more of the methodologies of the presently disclosed embodiments.

In general, the routines executed to implement the embodiments of the disclosure, may be implemented as part of an operating system or a specific application, component, program, object, module, or sequence of instructions referred to as “computer programs.” The computer programs typically comprise one or more instructions (e.g., instructions 804, 808, 828) set at various times in various memory and storage devices in a computer, and that, when read and executed by one or more processing units or processors 802, cause the computing system 800 to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include recordable type media such as volatile and non-volatile memory devices 810, floppy and other removable disks, hard disk drives, optical disks (e.g., Compact Disk Read-Only Memory (CD ROMS), Digital Versatile Disks (DVDs)), and transmission type media such as digital and analog communication links.

The network adapter 812 enables the computing system 800 to mediate data in a network 814 with an entity that is external to the computing system 800, such as a network appliance, through any known and/or convenient communications protocol supported by the computing system 800 and the external entity. The network adapter 812 can include one or more of a network adaptor card, a wireless network interface card, a router, an access point, a wireless router, a switch, a multilayer switch, a protocol converter, a gateway, a bridge, bridge router, a hub, a digital media receiver, and/or a repeater.

The network adapter 812 can include a firewall which can, in some embodiments, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including for example, the access and operation rights of an object by an individual, a machine, and/or an application, and the circumstances under which the permission rights stand.

Other network security functions can be performed or included in the functions of the firewall, including intrusion prevention, intrusion detection, next-generation firewall, personal firewall, etc.

As indicated above, the techniques introduced here implemented by, for example, programmable circuitry (e.g., one or more microprocessors), programmed with software and/or firmware, entirely in special-purpose hardwired (i.e., non-programmable) circuitry, or in a combination or such forms. Special-purpose circuitry can be in the form of, for example, one or more application-specific integrated circuits (ASICs), programmable logic devices (PLDs), field-programmable gate arrays (FPGAs), etc.

Note that any of the embodiments described above can be combined with another embodiment, except to the extent that it may be stated otherwise above or to the extent that any such embodiments might be mutually exclusive in function and/or structure.

Although the present invention has been described with reference to specific exemplary embodiments, it will be recognized that the invention is not limited to the embodiments described, but can be practiced with modification and alteration within the spirit and scope of the appended embodiments. Accordingly, the specification and drawings are to be regarded in an illustrative sense rather than a restrictive sense.

What is described includes:

1. A method performed by a server computer system of a simulated reality platform for rehabilitating users of the platform by promoting real-world behaviors of users engaged with simulated real-world experiences, the method comprising:
initiating a session for a simulated real-world experience including a real-world scene selected from a plurality of real-world scenes, the simulated real-world experience is configured to promote a real-world behavior of a user engaged with the simulated real-world experience;
receiving a plurality of inputs obtained during the simulated real-world experience, the received plurality of inputs include any of user responses to prompts, real-world positional data of the user, or real-world physiological data of the user;
causing a next real-world scene to render in the simulated real-world experience, the next real-world scene being selected based on at least some of the received plurality of inputs;
evaluating the user based on the received plurality of inputs processed in accordance with an expert system to predict the user's real-world behavior and identify a corresponding treatment;
responsive to determining that the user demonstrated corrected or improved behavior based on the evaluation of the user, awarding the user at least one of an opportunity to advance to another scene or level of the session or awarding redeemable points to the user.

2. The method of claim 1, further comprising:
executing machine learning based on the received plurality of inputs to improve the expert system for simulated real-world experiences that promote real-world behaviors and improve predicting real-world behaviors or identifying treatments.

3. The method of claim 2 further comprising:
improving selection of a next real-world scene to promote a real-world behavior of the user based on the machine learning.

4. The method of claim 2, wherein the machine learning is supervised by a third-party provider of the simulated real-world experience.

5. The method of claim 2, wherein the machine learning is unsupervised.

6. The method of claim 1, wherein the simulated real-world experience is an augmented reality simulation.

7. The method of claim 1, wherein the simulated real-world experience is a virtual reality simulation.

8. The method of claim 1, wherein the selected real-world scene is selected by the user engaged with the simulated real-world experience.

9. The method of claim 1, wherein the selected real-world scene is selected by a third-party provider of the simulated real-world experience.

10. The method of claim 1, wherein the expert system is a cognitive behavioral system.

11. The method of claim 1, wherein each real-world scene elicits selection by a user of one of a plurality of responses, and each next real-world scene increases in complexity with successful selection of a response to a previous real-world scene, wherein a more complex real-world scene has a greater number of selectable responses compared to a less complex real-world scene.

12. The method of claim 1, wherein at least some of the received plurality of inputs are anonymized to remove data identifying the user.

13. The method of claim 1, wherein evaluating a user is based on a time interval between two inputs or between a user's reaction to a stimuli.

14. The method of claim 1, wherein each real-world scene includes a stimuli configured to trigger a reaction by the user.

15. The method of claim 1, wherein the expert system is a cognitive behavioral system for rehabilitating the user.

16. The method of claim 1, wherein each real-world scene elicits an alternatively selectable predetermined response from the user.

17. The method of claim 1, wherein the real-world physiological data includes a plurality of blood pressure data, brain wave activity data, eye movement data, or heart electrical activity data.

18. The method of claim 1, wherein evaluating the user comprises:
comparing conscious user inputs to real-world physiological data.

19. The method of claim 1, wherein the treatment is a recommended therapeutic treatment identified from a plurality of therapeutic treatments.

20. The method of claim 1, wherein the real-world behavior is a behavior that mitigates a risk of recidivism.

21. The method of claim 1, wherein determining the next real-world scene comprises:
identifying a different real-world scene of the plurality of real-world scenes as that next real-world scene.

22. The method of claim 1, wherein the plurality of real-world scenes are rendered seamlessly to promote the real-world behavior by the user.

23. The method of claim 1, further comprising:
outputting data indicative of at least one of the evaluation, the predicted user's real-world behavior, or a recommendation for a treatment.

24. The method of claim 1, wherein the expert system implements artificial intelligence to evaluate the user.

25. The method of claim 1, wherein the server computer system is a cloud-based computer system.

26. A server computer system of a simulated reality platform for rehabilitating users of the platform by promoting real-world behaviors of users engaged with simulations, comprising:
one or more processor; and
one or more memories including instructions executable by the processors causing the server computer system to:
initiate an augmented reality or virtual reality simulation of a real-world scene selected from a plurality of real-world scenes, the selected real-world scene is configured to promote a real-world behavior of a user engaged with the simulation;
receive a plurality of inputs obtained during the simulation, the received plurality of inputs including user responses to prompts, real-world positional data of the user, and real-world physiological data;
cause a next real-world scene to render in the simulation, the next real-world scene being selected based on at least some of the received plurality of inputs;
evaluate the user based on the received plurality of inputs processed with an expert system to predict the user's real-world behavior and identify a corresponding treatment;
output data indicative of at least one of the evaluation, the predicted user's real-world behavior, or the treatment; and
execute machine learning based on the received plurality of inputs to improve the expert system for simulations that promote real-world behaviors and identifying treatments or predicting real-world behaviors.

27. A computer system comprising:
one or more processors; and
one or more memories including instructions executable by the processors causing the computer system to:
load an augmented reality or virtual reality simulation of a real-world scene selected from a plurality of real-world scenes, the selected real-world scene configured to promote a real-world behavior of a user engaged with the simulation;
receive a plurality of inputs obtained during the simulation, the received plurality of inputs including user responses to prompts, real-world positional data of the user, and real-world physiological data;
send at least some of the received plurality of inputs over a computer network to a server computer system configured to:
enable selection of a next real-world scene by a third-party provider of the simulation to promote the user's real-world behavior,
evaluate the user with an expert system to predict the user's real-world behavior and identify a corresponding treatment,
output data indicative of the evaluation, the predicted user's real-world behavior, or the treatment, and
execute machine learning to improve the expert system to promote real-world behaviors, predict real-world behaviors of any users, or identify treatments; and
load the next real-world scene in the selected real-world scene of the simulation to promote the real-world behavior of the user.

28. A method performed by a user computer of a simulated reality platform for simulating real-world scenes to promote a real-world behavior of a user immersed in a simulation, the method comprising:
initiating a session for a real-world simulation including a real-world scene selected from a plurality of real-world scenes of the real-world simulation;
receiving an authentication code to enable the session for the user of the real-world simulation to experience the selected real-world scene; and
upon successful authentication of the user based on the authentication code, launching the session to render the real-world simulation including the selected real-world scene for the user as authorized by the authentication code.

29. The method of claim 28, wherein receiving the authentication code comprises:
receiving the authentication code over a communications channel different from the communications channel used to request initiating the session.

30. The method of claim 28, wherein the authentication code is a multi-digit passcode.

31. The method of claim 28, wherein the selected real-world scene is selected autonomously and without user intervention or selected by an administrator for the user.

32. The method of claim 28, wherein the selected real-world scene is selected by the user of the simulated reality platform.

33. The method of claim 28, wherein the authentication code authenticates the user and authenticates the selected real-world scene.

34. The method of claim 28, wherein the authentication code authenticates only the session.

35. The method of claim 28, wherein the plurality of real-world scenes is a sequence of ordered real-world scenes and the selected real-world scene is authenticated only if any prior real-world scenes of the sequence have been successfully completed by the user.

36. A method performed by a client computer administering a simulated reality by a user device to promote a real-world behavior of a user engaged with a simulation, the method comprising:
connecting the client subsystem to a cloud service by calling an application programming interface (API) of the cloud service to grant access to a simulated reality platform such that the client computer administers a session including a simulation of a real-world scene configured to promote a real-world behavior of the user engaged with the simulation;
causing the user computer to render the simulation of the real-world scene under the control of the client computer and in accordance with an authorization granted by the cloud service.

37. The method of claim 36 further comprising, prior to causing the user computer to render the simulation:
authenticating the session for the user computer to render the simulation.

38. The method of claim 36, wherein the real-world scene is included in a sequence of ordered real-world scenes for the session.

39. The method of claim 36 further comprising:
collecting data of the simulation in real-time, the data indicating inputs by a user of the client computer in response to the simulation; and
determining whether the user is being successfully rehabilitated based on the collected data.

40. A method performed by one or more server computers of a simulated reality platform for administering a simulation to promote a real-world behavior of a user engaged with the simulation, the method comprising:
creating a plurality of simulations that can promote one or more real-world behaviors by a user, each simulation including a plurality of scenes;
creating a user profile including information indicating an ailment of the user for which the user seeks rehabilitation;
identifying one or more simulations including a plurality of scenes to promote the desired real-world behavior to rehabilitate the user;
linking the user profile to the one or more identified simulations;
enabling the simulation capable of rehabilitating the user, the simulation including a course for traversing through a subset of the plurality of scenes; and
adjusting the course to traverse a different subset of the plurality of scenes in response to the user failing to demonstrate the desired real-world behavior.

41. The method of claim 40, wherein the user profile and the course are stored in a database, the method further comprising:
modifying a user profile stored in the database to reflect the adjusted course, wherein the failure to demonstrate the desired real-world behavior is based on a measure of progress by the user to demonstrate the real-world behavior by the user.

42. The method of claim 40, wherein a scene is selected for a user based on the user's profile.

43. The method of claim 40, wherein the course is enabled for the user in response to a payment made by the user for that course.

44. The method of claim 40 further comprising:
receiving data collected by a plurality of client computers administering a plurality of simulations for a plurality of user subsystems running the simulations;
applying machine learning processes on the collected data to learn about simulations, content of scenes, or courses traversing the scenes; and
modifying the simulations based on an outcome of the machine learning.

45. A simulated reality platform for rehabilitating users of the platform by promoting real-world behaviors of users engaged with simulations, the computer system comprising:
a cloud subsystem configured to create and store a library of simulations each including a set of scenes configured to promote real-world behaviors of users engaged with simulations;
a client subsystem configured to administer a particular simulation provided by the cloud subsystem for a particular user such that the user experiences a course of a subset of a set of scenes; and
a user subsystem including a head mounted near-to-eye display operable to render the subset of scenes as administered by the client subsystem to promote a real-world behavior of the user engaged in the simulation.

46. The simulated reality platform of claim 45, wherein the user subsystem further comprises:
one or more sensors configured to detect the user's movement while the user is engaged in a simulation and provide feedback to the client subsystem administering the simulation for that user.

47. The simulated reality platform of claim 46, wherein the user subsystem further comprises:
one or more handheld electronic wands configured to receive input from the user engaged in the simulation.

48. A head mounted display system comprising:
a chassis;
at least one display mounted to the chassis, to render a scene of a simulated reality for an optical receptor of a user when the head mounted display system is worn by the user, the simulation including a plurality of scenes configured to promote a real-world behavior by the user wearing the head mounted display system; and
a camera mounted to the chassis, to capture movement of the optical receptor responsive to the plurality of scenes; and
a network interface configured to communicate with a client subsystem configured to administer the simulation.

49. The computer system of claim 48, wherein a scene is rendered by the at least one display to overlay a real-world view by the optical receptor of the user.

50. The computer system of claim 48, wherein a scene is rendered on the at least one display to create a virtual reality view by the optical receptor of the user.
